(19) United States
(12) Patent Application Publication
(10) Pub. No.: US 2016/0376632 A1
(43) Pub. Date: Dec. 29, 2016

(54) KINASE ACTIVITY DETECTION METHODS

(71) Applicants: REGENTS OF THE UNIVERSITY OF MINNESOTA, Minneapolis, MN (US); PURDUE RESEARCH FOUNDATION, West Lafayette, IN (US)

(72) Inventors: Laurie L. Parker, Minneapolis, MN (US); Andrew M. Lipchik, Minneapolis, MN (US)

(21) Appl. No.: 15/195,712

(22) Filed: Jun. 28, 2016

Related U.S. Application Data
(63) Continuation-in-part of application No. 14/750,916, filed on Jun. 25, 2015.
(60) Provisional application No. 62/016,994, filed on Jun. 25, 2014.

(51) Int. Cl. C12Q 1/48

(52) U.S. Cl. CPC C12Q 1/485; G01N 2333/91205; G01N 2458/40

(57) ABSTRACT

The invention provides a method for detecting the activities of two or more kinases. The method enables multiplexed detection with high signal to noise in a high-throughput-compatible format and a platform that could be applied to other lanthanide metal and fluorophore combinations to achieve even greater multiplexing without the need for phosphospecific antibodies.

### PRIORITY OF INVENTION

This application is continuation in part of U.S. application Ser. No. 14/750,916, filed Jun. 25, 2015, which claims priority from U.S. Provisional Application Ser. No. 62/016,994, filed Jun. 25, 2014. The entire content of the applications referenced above are hereby incorporated by reference herein.

### GOVERNMENT FUNDING

This invention was made with government support under grants CA127161 and CA182543 awarded by the National Institutes of Health. The United States Government has certain rights in this invention.

### BACKGROUND

Kinase signalling is a major mechanism driving many cancers. While many inhibitors have been developed and are employed in the clinic, resistance due to crosstalk and pathway reprogramming is an emerging problem. High-throughput assays to detect multiple pathway kinases simultaneously could better model these complex relationships and enable drug development to combat this type of resistance.

Numerous leukemias and lymphomas have been characterized by the clonal expansion of B-lymphocytes due to the deregulation of the B-cell receptor-signalling pathway (Kuppers, R. Nat Rev Cancer 2005, 5, 251; and Nogai, HxyH. W., et al., Anal Chem., 2011, 83, 9687). Förster resonance energy transfer (FRET) based assays have been developed to monitor multiple dynamic cellular processes simultaneously in a single assay (Peyker, A., et al., Chembiochem. 2005, 6, 78; Galperin, E., et al., Nat Methods, 2004, 1, 209; Kienzler, A., et al., Bioconjug Chem., 2011, 22, 1852; Piljic, A.; Schultz, C. ACS Chem. Biol, 2008, 3, 156; and Ding, Y., et al., Anal. Chem., 2011, 83, 9687). However, while useful in some applications, FRET based methods that use organic fluorophores or fluorescent proteins as both the donor and acceptor suffer from limitations including small dynamic ranges, small Stokes shifts, often having wide emission peaks that can result in spectral bleed through, and the requirement for genetic engineering and expression of protein fluorophores.

Tyrosine kinases Lyn (a Src family kinase), spleen tyrosine kinase (Syk) and Bruton's tyrosine kinase (Btk) are the main signal transducers in this pathway. Thus, they have become popular therapeutic targets for small molecule inhibitors (Mahadevan, D., Fisher, R. I., J Clin. Oncol, 2011, 29, 1876). Despite the identification of this pathway as a cause of disease, effective therapeutic options targeting the B-cell receptor pathway and/or these kinases are still relatively limited. Often these kinase activities are dependent on each other, which can affect the efficacy of inhibitor drugs targeting individual enzymes.

Lanthanides (Ln³⁺) have been explored as probes in biological assays for the detection of ligand binding, enzyme activity, and protein-protein interactions due to their unique optical properties (Hermanson, S. B., et al., PLoS One, 2012, 7, e43580; Jeyakumar, M., et al., Biochemistry, 2008, 47, 7465; Jeyakumar, M., Katzenellenbogen, J. A., Anal Biochem, 2009, 386, 73; Rajapakse, H. E., et al., Proc Natl Acad Sci USA, 2010, 107, 13582; Sculimbrene, B. R.; Imperiali, B. J Am Chem Soc, 2006, 128, 7346; Vuojola, J., et al., Anal Chem, 2013, 85, 1367; Weitz, E. A., et al., J Am Chem Soc, 2012, 134, 16099; Yapici, E., et al., Chembiochem, 2012, 13, 553; and Hildebrandt, N., et al., Coordination Chemistry Reviews, 2014, 273, 125).

Compared to organic fluorophores and fluorescent proteins, the Lanthanides usually have narrow emission bands, large Stokes shifts, and long photoluminescence lifetimes. This can enable time-resolved analysis, high sensitivity and specificity of detection due to reduced interference from short-lived background fluorescence. These also allow multiplexed detection via the multiple distinct, well-resolved emission bands that can be exploited for luminescence resonance energy transfer (LRET) to more than one acceptor fluorophore. Previously, development of peptide biosensors capable of detecting tyrosine kinase activity through phosphorylation-enhanced terbium (Tb³⁺) luminescence has been described (Lipchik, A. M., Parker, L. L., Anal Chem, 2013, 85, 2582; Lipchik, A. M., et al., J Am Chem Soc, 2015, 137, 2484; and Cui, W., Parker, L. L., Chem Commun (Camb), 2015, 51, 362).

Multiplexed kinase activity detection has remained a challenge in the field, with only a few examples of successful implementation. Existing examples of this strategy typically use dual antibody labelling, with one antibody tagged with a small molecule fluorophore for emission and the other labelled with a chelated lanthanide for sensitization. Alternatively, existing examples tag the substrate with a fluorophore (either small molecule or protein) and use a phosphospecific antibody labelled with a chelated lanthanide for sensitization. In either case, highly specific antibodies are required (but may not be available for the desired analytes) to enable multiplexing.

There is currently a need for new detection strategies that offer sensitive and specific detection of multiple kinase activities that can enhance the depth of information obtained in a screening assay, monitor more than one signal simultaneously and mimic reconstitution of the relevant pathways, without relying on the availability or development of antibodies for detection.

### SUMMARY

The present invention provides a strategy to take advantage of time-resolved luminescence of Lanthanide-associated peptides, which facilitate efficient energy transfer to small molecule fluorophores conjugated to the peptides to produce orthogonally-colored biosensors. The methods of the invention enable multiplexed detection with high signal to noise in a high-throughput-compatible format. This provides a platform that can be applied to other lanthanide metal and fluorophore combinations to achieve even greater multiplexing without the need for phosphospecific antibodies.

Accordingly, in one aspect the invention provides a method for detecting the activities of two or more kinases comprising:
a) contacting a first kinase and a second kinase with a first peptide and a second peptide, wherein:
   i) the first peptide is a substrate for the first kinase;
   ii) the second peptide is a substrate for the second kinase;
   iii) each peptide is associated with a lanthanide;
   iv) each peptide comprises a group capable of sensitizing the lanthanide that is associated with that peptide; and
   v) each peptide is linked to a fluorophore
      under conditions such that a first signal associated with the activity of the first kinase and a second signal that is associated with the activity of the second kinase are generated; and
b) detecting the first signal and the second signal.

In another aspect, the development of a platform for detection of kinase activity that leverages the overlap of the multiple distinct emission bands of lanthanides (e.g. Tb³⁺) with orthogonal fluorescently labeled peptide substrates that are capable of phosphorylation-enhanced lanthanide (e.g. Tb³⁺) luminescence is provided.

In another aspect, a method for simultaneously or consecutively detecting at least two kinase activities either simultaneously or consecutively is provided. In one aspect, the method uses a Förster resonance energy transfer (FRET). Preferably, the donor fluorophore has a narrow emission band. Also, preferably, the donor fluorophore has a large Stokes shift.

In another aspect, the methods include multiplexed detection via the multiple distinct, well-resolved emission bands of the donor fluorophore that can be exploited for luminescence resonance energy transfer (LRET) to more than one acceptor fluorophore.

The methods of the invention circumvent some of the limitations of antibody-based TR-FRET/LRET approaches and complement previous strategies, enabling direct sensing of phosphate incorporation to the biosensors, avoiding the need for antibody labels and streamlining the path from enzyme reaction to assay read-out. This strategy is compatible with a variety of kinases and fluorophores to increase the number of activities monitored in a single reaction, setting the stage for pathway-based drug screening to target signalling pathway reprogramming in inhibitor resistance.

### BRIEF DESCRIPTION OF THE FIGURES

FIGS. 1A-C illustrate multiplexed detection using time-resolved lanthanide-based resonance energy transfer (TR-LRET) and fluorophore conjugated peptide biosensors. FIG. 1A illustrates an emission spectrum of phosphopeptide-Tb³⁺ complex (black), excitation (dashed lines) and emission (solid lines) spectra of the two acceptor fluorophores 5-FAM (G-green) and Cy5 (R-red). FIG. 1B illustrates schematic TR-LRET detection of Lyn tyrosine kinase activities using the 5-FAM-SFAStide-A (5-FAM-Ahx-GGEEDEDIYEELDEPGGKbiotinGG) biosensor. FIG. 1C illustrates schematic TR-LRET detection of Syk tyrosine kinase activities using the SAStide-Cy5 (GGDEEDYEEDEPGGCCy5GG) biosensor.

FIGS. 2A-B illustrate Time-Resolved Lanthanide-based Resonance Energy Transfer (TR-LRET) detection of phosphorylation-dependent signals and fluorescence cross-interference. FIG. 2A illustrates time-resolved luminescence emission spectra for SAStide-Cy5 (dashed line) and pSAStide-Cy5 (solid line). FIG. 2B illustrates the 5-FAM-SFAStide-A (dashed line) and 5-FAM-pSFAStide-A (solid line). Spectra were collected from 15 μM peptide in the presence of 100 μM Tb3+ in 10 mM HEPES, 100 mM NaCl, pH 7.5, λex=266 nm, in 50 μL total volume, 1 ms collection time, 50 μs delay time, and sensitivity 180. Data represent the average of experiments performed in triplicate.

FIGS. 3A-F illustrate Simultaneous multiplexed in vitro detection of Syk and Lyn kinase activities. FIG. 3A illustrates the in vitro Lyn assay luminescence emission spectra in the presence of both 5-FAM-SFAStide-A and SAStide-Cy5. FIG. 3C illustrates in vitro Syk assay luminescence emission spectra in the presence of both 5-FAM-SFAStide-A and SAStide-Cy5. FIG. 3E illustrates in vitro Lyn and Syk assay luminescence emission spectra the presence of both 5-FAM-SFAStide-A and SAStide-Cy5. FIGS. 3B, 3D, and 3F illustrate the quantification of 5-FAM-SFAStide-A signal and SAStide-Cy5 signal for each assay. Assays were performed in the presence of 2.5 μM 5-FAM-SFAStide-A, 12.5 μM SAStide-Cy5, Lyn, Syk or both kinases (15 nM), 100 μM ATP, 10 mM MgCl₂ and ng/μL BSA.

FIG. 4 illustrates a synopsis of the transition of the excitation process for the lanthanide complexes, from low excitation and energy transfer when the peptides are unphosphorylated, to higher excitation and energy transfer when the peptides are phosphorylated.

FIGS. 5A-C, 6A-C, 7A-C, and 8A-C illustrate the peptide characterization using high-performance liquid chromatography/mass spectrometry (HPLC-MS) analysis to measure molecular weight (via mass-to-charge ratio, m/z) and UV absorbance at 214 nm (typical for molecular characterization of peptides). FIGS. 5A, 5B, and 5C, are for SAStide-Cy5 (GGDEEDYEEPDEPGGC_{Cy5G}G); FIGS. 6A, 6B, and 6C, are for pSAStide-Cy5 (GGDEEDYEEPDEPGGC_{Cy5}GG); FIGS. 7A, 7B, and 7C, are for 5-FAM-SFAStide-A (5-FAMAhxGGEEDEDIYEELDEPGGK_{biotin}GG); and FIGS. 8A, 8B, and 8C, are for 5-FAM-pSFAStide-A (5-FAMAhxGGEEDEDIYEELDEPGGK_{biotin}GG).

FIGS. 9A-B illustrate the luminescent properties of pSAStide-Cy5 and 5-FAM-pSFAStide-A. Luminescence excitation spectra for pSAStide-Cy5 (FIG. 9A) and 5-FAM-pSFAStide-A (FIG. 9B) were collected in the presence (P) or absence (A) of Tb³⁺. Emission at the respective λₘₐₓ for each organic fluorophore (Y-axis) was measured at the excitation wavelengths across the range for tyrosine absorbance (shown on the X-axis). While Cy5 showed no excitation in the absence of Tb³⁺ (indicating complete Tb³⁺-dependence), 5-FAM showed some background excitation both in the absence and presence of Tb³⁺, however at a higher wavelength than is used in the typical LRET biosensor assay (266 nm). Emission maxima were collected from 15 μM peptide in the presence of 100 μM Tb³⁺ or absence, 10 mM HEPES, 100 mM NaCl with a 50 μs delay and 1 ms collection time. Each spectrum represents the average of three replicates.

FIGS. 10A-B illustrate the quantification of LRET-dependent fluorophore signal. Quantification of the fluorophore signal was accomplished for SAStide-Cy5 (FIG. 10A) and 5-FAM-SFAStide-A (FIG. 10B) by fitting a Gaussian curve to the individual signals and integrating the curve.

FIGS. 11A-B illustrate pSAStide-Cy5 cross-interference with SFAStide-A-5-FAM signal (FIG. 11A) and pSFAStide-A-5-FAM cross-interference with SAStide-Cy5 signal (FIG. 11B). Spectra were collected from 0.5 μM SFAStide-A-5-FAM and 2.5 μM SAStide-Cy5 in the presence of 10 μM Tb³⁺ in 10 mM HEPES, 100 mM NaCl, pH 7.5, 1.2 M Urea, 20 μM ATP, 0.2 ng/μL BSA, 2 mM MgCl₂, λₑₓ=266 nm, in 100 μL total volume, 1 ms collection time, 100 μs delay time, and sensitivity 180. Data represent the average of experiments performed in triplicate.

FIGS. 12A-C illustrate the Luminescence decay rates for the peptide biosensor-Tb³⁺ complexes with and without fluorophore conjugation.

FIGS. 13A-D illustrate the TR-LRET quantitative detection of biosensor phosphorylation. FIG. 13A illustrates pSAStide-Cy5-Tb³⁺ emission spectra with increasing proportions of phosphorylated biosensor compared to unphosphorylated in the presence of unphosphorylated 5-FAM-SFAStide-A. FIG. 13B illustrates Cy5 emission spectral area calibration curve based on spectra from (FIG. 13A) and the integrated area of the Cy5 emission peak. FIG. 13C illustrates 5-FAM-pSFAStide-A-Tb³⁺ emission spectra at increasing proportions of phosphorylated biosensor compared to unphosphorylated in the presence of unphosphorylated SAStide-Cy5. FIG. 13D illustrates 5-FAM emission spectral area calibration curve based on (FIG. 13C). Spectra were collected from 0.5 μM SFAStide-A-5-FAM and 2.5 μM SAStide-Cy5 in the presence of 10 μM Tb³⁺ in 10 mM HEPES, 100 mM NaCl, pH 7.5, 6 M Urea, 100 μM ATP, 12.5 μg/μL BSA, 10 mM MgCl₂, λₑₓ=266 nm, in 100 μL total volume, 1 ms collection time, 100 μs delay time, and sensitivity 180. Data represent the average of experiments performed in triplicate, error bars in the AUC plots represent SEM.

FIG. 14 illustrates the validation of in vitro specificity of SAStide-Cy5 and 5-FAM-SFAStide-A using ELISA-based chemifluorescence. The SAStide biosensor was incubated with Syk-EGFP and the 5-FAM-SFAStide-A biosensor with Lyn in an in vitro kinase assay as described in the main text. Aliquots were removed at designated time points, quenched with EDTA and alongside the TR-LRET detection as described in FIG. 3, the amount of phosphorylated substrate was also measured using ELISA-based detection.

FIGS. 15A-C illustrate the formation of QD-biosensor conjugates used in this study was evaluated by electrophoresis on 1% agarose gel. FIG. 15A illustrates QD605-SAStide conjugate. Lanes (left to right): QD/pSAStide ratio 1:0, 1:50, 1:100, 1:200, 1:300, QD/SAStide ratio 1:50, 1:100, 1:200, 1:300. FIG. 15B illustrates QD655-SFAS-A conjugate. Lanes (left to right): QD/pSFAStide-A ratio 1:0, 1:50, 1:100, 1:200, 1:300, QD/SFAStide-A ratio 1:50, 1:100, 1:200, 1:300. FIG. 15C illustrates QD655-SFAS-A conjugate with the presence of 2.4M urea (the quenching reagent used in kinase assay). Lanes (left to right): QD/SFAStide-A (without urea) ratio 1:0, 1:50, 1:100, 1:200, QD/SFAStide-A (with urea) ratio 1:50, 1:100, 1:200.

FIG. 16 illustrates the schematic mechanism of time-resolved LRET detection using phosphorylated QD-biosensor conjugate. Phosphorylated biosensor leads to stronger time-resolved LRET emission from the conjugated QD, whereas unphosphorylated biosensor leads to weak LRET emission. Actual conjugates have multiple biosensors bound to QD surface.

FIGS. 17A-E illustrate Time-resolved LRET emission of QD-biosensor conjugates. FIG. 17A illustrates the emission spectra comparing steady-state and time-resolved emission of the QD-biosensor conjugates used in this study. Steady state emission from QD605-pSAStide (10 nM) and QD655-pSFAStide-A (10 nM) conjugate are dominated by inherent QD emission. However, time-resolved LRET emission from both conjugates showed significant difference between emission spectra of the phospho- and unphospho-conjugates. FIG. 17B illustrates that in order to mimic the experimental condition of kinase assay, a calibration curve for QD605-SAStide was established in kinase reaction buffer. FIG. 17C illustrates that in order to mimic the experimental condition of multiplexed kinase assay, a calibration curve for QD655-SFAStide-A was established in kinase reaction buffer. FIG. 17D illustrates the emission spectra of samples from (FIG. 17B) showing that TR-LRET emission increased as the percent of pSAStide (proportional to total biosensor) increased. FIG. 17E illustrates the emission spectra of samples from (FIG. 17C) showing that TR-LRET emission increased as the percent of pSFAStide-A (proportional to total biosensor) increased. For (FIG. 17B) and (FIG. 17C), the QD-biosensor conjugates were diluted to 20 nM with 100 μM Tb³⁺ in 10 mM HEPES buffer (pH=7.5) and other kinase reaction reagents. Time-resolved emission spectra were recorded using built-in monochromator on a Synergy 4 microplate reader with 250 μs delay and 1000 μs integration. Excitation wavelength: 266 nm. Calibration curves were established using luminescence reading from a 605/10 and a 655/10 filter accordingly with a 265/10 excitation filter.

FIGS. 18A-H illustrate multiplexed kinase assay demonstrated by using two different QD-biosensor conjugates (final concentration: 20 nM). In general, kinase assays were performed with 15 nM kinase, 5 μM peptides, 50 nM QD, 100 μM ATP, 10 mM Mg²⁺, and 0.2 μg/μL BSA in 25 mM HEPES buffer. Aliquots of samples were quenched in detection buffer (40 μL 6 M urea, 10 μL 1 M NaCl, 10 μL 1 mM Tb³⁺) as detailed in supporting information. TR-LRET emission was quantified by corresponding calibration curves (FIG. 17B, 17C). FIG. 18A illustrates time-resolved spectra of QD605-SAStide conjugate from Syk kinase assay at 1 min, 15 min, and 45 min after addition of Syk. Conjugate was formed after kinase assay, and 1 μM Na₃VO₄ was also included. FIG. 18B illustrates time-resolved spectra of pre-formed QD605-SAStide and QD655-SFAStide-A conjugates from Syk kinase assay at 1 min, 15 min, and 30 min after addition of Syk. Delay time was set to 400 μs to minimize cross-interference between conjugates. FIG. 18C illustrates quantification of QD605-SAStide phosphorylation from (FIG. 18A) at 1 min, 15 min, and 45 min after addition of Syk. FIG. 18D illustrates quantification of QD605-SAStide and QD655-SFAStide-A phosphorylation by Syk from (FIG. 18B) at 1 min, 15 min, and 30 min after addition of Syk. Specific QD605-SAStide phosphorylation by Syk is shown. Horizontal dashed line, 0% phosphorylation. Dashed line, QD605-SAStide signal. Dotted line, QD655-SFAStide-A signal. FIG. 18E illustrates time-resolved spectra of QD655-SFAStide-A conjugate from Src kinase assay at 1 min, 5 min, and 15 min after addition of Src. Conjugates were formed before kinase assay. FIG. 18F illustrates time-resolved spectra of pre-formed QD605-SAStide and QD655-SFAStide-A conjugates from Src kinase assay at 1 min, 5 min, and 15 min after addition of Src. FIG. 18G illustrates quantification of QD655-SFAStide-A phosphorylation from (FIG. 18E) at 1 min, 5 min, and 15 min after addition of Src. FIG. 18H illustrates quantification of QD605-SAStide and QD655-SFAStide-A phosphorylation by Src from (FIG. 18F) at 1 min, 5 min, and 15 min after addition of Src. Specific QD655-SFAStide-A phosphorylation by Src is shown. Horizontal dashed line, 0% phosphorylation. Dashed line, QD655-SFAStide-A signal. Dotted line, QD605-SAStide signal.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

The term “narrow emission band” means that the emission range for each distinct emission maximum (of which lanthanides typically have more than one) will be about 15 nm to about 40 nm, preferably the emission range will be about 20 nm to about 30 nm.

The term “large Stokes shift” means that the Stokes Shift for the complex is from about 266 nm excitation to about 450-680 nm emission.

Aryl denotes a phenyl radical or an ortho-fused bicyclic carbocyclic radical having about nine to ten ring atoms in which at least one ring is aromatic. Heteroaryl encompasses a radical of a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₄)alkyl, phenyl or benzyl, as well as a radical of an ortho-fused bicyclic heterocycle of about eight to ten ring atoms comprising one to four heteroatoms each selected from the group consisting of non-peroxide oxygen, sulfur, and N(X).

The term “amino acid,” comprises the residues of the natural amino acids (e.g. Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g. phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, α-methyl-alanine, para-benzoylphenylalanine, phenylglycine, propargylglycine, sarcosine, and tert-butylglycine). The term also comprises natural and unnatural amino acids bearing a conventional amino protecting group (e.g. acetyl or benzyloxycarbonyl), as well as natural and unnatural amino acids protected at the carboxy terminus (e.g. as a (C₁-C₆)alkyl, phenyl or benzyl ester or amide; or as an α-methylbenzyl amide). Other suitable amino and carboxy protecting groups are known to those skilled in the art (See for example, T. W. Greene, Protecting Groups In Organic Synthesis; Wiley: New York, 1981, and references cited therein).

### Peptides

“Peptide” describes a sequence of 2 to 50 amino acids or peptidyl residues. The sequence may be linear or cyclic. A peptide can be linked to a fluorophore or to a chelating group through the carboxy terminus, the amino terminus, or through any other convenient point of attachment, such as, for example, through the sulfur of a Cysteine.

The peptides used in the methods of the invention: 1) are each a substrate for a kinase, 2) are capable of associating with a lanthanide metal either through hydrostatic interactions or through a group capable of chelating the lanthanide, 3) comprise a group that is capable of sensitizing the associated lanthanide metal, and 4) are linked covalently either directly or through a linking group to a fluorophore that can be sensitized by the lanthanide metal.

Typically, the group that is capable of sensitizing the associated lanthanide metal includes an aryl or a heteroaryl ring. In one aspect, the group that is capable of sensitizing the associated lanthanide metal may be an aromatic ring in an amino acid of the peptide. Non-limiting examples of amino acids having an aromatic ring include tyrosine, histidine, phenylalanine, and tryptophan. Preferred amino acids are tyrosine and tryptophan. A more preferred amino acid is tyrosine. The peptide can be any size. Preferably, the peptide comprises from about 3 to about 40 amino acids, preferably from about 5 to about 25 amino acids and more preferably, about 18 amino acids. Typically, the peptide is 1) a substrate for at least one kinase, 2) able to associate with a lanthanide, 3) capable of sensitizing the lanthanide and 4) linked to a fluorophore.

Suitable peptides can be prepared using methods known in the art. For example, they can be prepared using methods similar to those described in United States Patent Application Publication Number US2013/0231265. They can also be prepared using methods similar to those described in and described U.S. Pat. Nos. 4,612,302; 4,853,371; and 4,684,620, and in published U.S. Patent Application Nos. 2014/0072516 A1 and 2013/0231265 A1 and as described in the Examples herein. Peptide sequences specifically recited herein are written with the amino terminus on the left and the carboxy terminus on the right.

Specific peptide-fluorophores that are substrates for the kinase shown are illustrated in Table 1.

TABLE 1
Peptide biosensor sequences^{[a][b]}

| Name | Kinase | Sequence |
|---|---|---|
| 5-FAM- | Src- | 5-FAM-Ahx-GGEEDEDIYE |
| SFAStide-A | family | ELDEPGGK_{b}GG |
| SAStide-Cy5 | Syk | GGDEEDYEEPDEPGGC_{Cy5}GG |

^{[a]}5-FAM = 5-carboxyfluorescein;Ahx = 6-aminohexanoic acid; Kb = biotinyl-L-lysine; C_{Cy5} = cysteine thiol conjugated with Cy5.
^{[b]}Sequence segments represented in bold are the core kinase recognition/Tb³⁺-chelation residues of the biosensor.

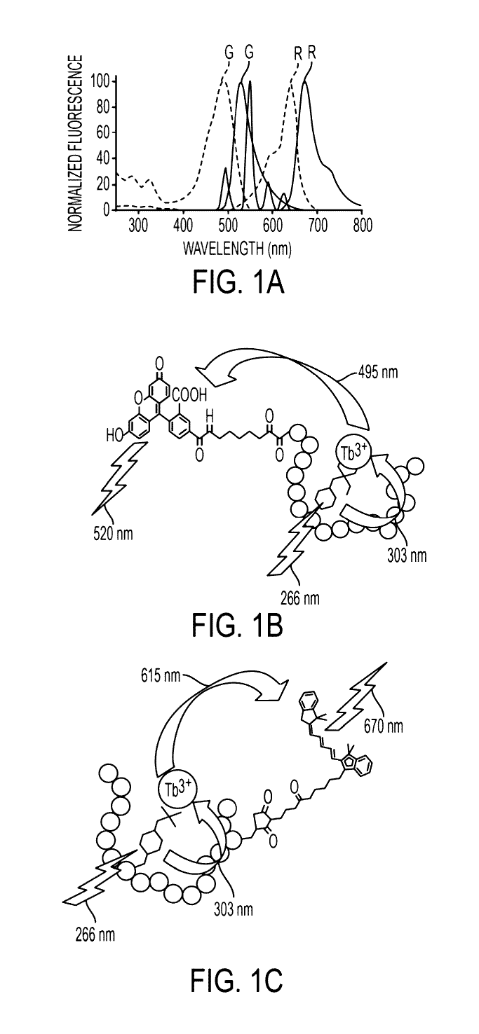

Compared to organic fluorophores and fluorescent proteins, the lanthanide-complexed peptide-fluorophores have narrow emission bands from about 15 to about 40 nm wide for each distinct emission maximum, large Stokes shifts (about 180 nm to about 450 nm shift), and long photoluminescence lifetimes (between about 50 microseconds and about 10 milliseconds), enabling time-resolved analysis, high sensitivity and specificity of detection due to reduced interference from short-lived background fluorescence. These improvements also allow multiplexed detection via the multiple distinct, well-resolved emission bands that can be exploited for luminescence resonance energy transfer (LRET) to more than one acceptor fluorophore. The bands are chosen such that the emission profiles do not overlap (e.g. FIG. 1A).

Previous kinase assay methods typically relied on antibodies for detection, with either the substrate or a substrate-specific antibody tagged with a small molecule fluorophore for emission, and a phosphospecific antibody labeled with a chelated lanthanide for detecting phosphorylation via donation to the small molecule fluorophore ((Hildebrandt, N., et al., Coordination Chemistry Reviews, 2014, 273, 125; Kim, S. H., et al., J Am Chem Soc, 2010, 132, 4685; Horton, R. A., Vogel, K. W., J Biomol Screen, 2010, 15, 1008; Kupcho, K. R., et al., J Am Chem Soc, 2007, 129, 13372). These previous methods were therefore limited to the antibodies available for a given substrate modification, and subject to the handling issues presented by such immunodetection workflows. The methods of the present invention have the advantage of not being similarly limited.

### Kinases

The methods of the invention can be used to assess the activity of any kinase for which a phosphorylation-dependent lanthanide sensitizing peptide substrate is available or can be prepared (see, Akiba, H. et al., Anal Chem. 2015 87(7):3834-40). One specific group of kinases is tyrosine kinases, serine kinases and threonine kinases. Another specific group of kinases is the Src-family kinases, Abl-family kinases, and Syk-family kinases. A more specific kinase is a kinase selected from the group consisting of the Src family (Lyn, Src, Hck, Fyn, Fgr, Lek), the JAK family (JAK1, JAK2, JAK3), the Abl family (Abl, Arg), and the Syk family (Zap-70, Syk).

### Lanthanides

The lanthanide or lanthanoid series of chemical elements (La, Ce, Pr, Nd, Pm, Sm, Eu, Gd, Tb, Dy, Ho, Er, Tm, Yb, and Lu) comprise the fifteen metallic chemical elements with atomic numbers 57 through 71, from lanthanum (La) through lutetium (Lu). These fifteen lanthanide elements, along with the chemically similar elements scandium and yttrium, are often collectively known as the rare earth elements and are suitable for the disclosed method. When in the form of coordination complexes, lanthanides are found usually in their +3 oxidation state. Suitable preferred lanthanides include Tb³⁺ and Eu³⁺, Sm³⁺, Dy³⁺, and Yb³⁺.

The lanthanides can be associated with the peptides through electrostatic interactions or they can be associated with a chelating group that is linked to the peptide directly or through a linking group. Non-limiting examples of suitable chelating groups can be found in Akiba, H. et al., Anal Chem. 2015 87(7):3834-40, and/or Tremblay, M. S. et al., Org Lett. 2006, 8(13):2723-6.

### Detection

The signal from phosphorylation of the biosensors can be detected with any fluorimeter, luminometer, or other spectroscopic detection device that is capable of excitation at the appropriate wavelength for the lanthanide-sensitizing moiety (for example tyrosine, tryptophan or other aromatic groups on a chelating ligand from about 200 to about 400 nm) and measuring emission at the appropriate wavelengths for the desired acceptor fluorophore signals (for example, typical small molecule fluorophores emitting between about 350 nm and 900 nm). Preferably, the detection device will be capable of time-resolved measurements, in which pulsed excitation is used and a time gate is employed to decrease background emission from non-lanthanide-sensitized fluorophores (which typically decay within nanoseconds). Such instrumentation will be well known to those skilled in the art, and include sample introduction formats such as cuvette-based, flow-based, microplate-based, and tube-based sample holding.

### Fluorophores

The fluorophores are typically chosen such that the emission profiles do not overlap (e.g., FIG. 1A).

It is noted that any fluorophore having a suitable overlap of excitation with the emission of a lanthanide will work in the invention. For example, 5-FAM was selected as the acceptor to couple with the pSFAStide-A-Tb³⁺ complex because it has a broad excitation peak at 495 nm that matches well with the ⁵D₄→⁷F₆ emission band of Tb³⁺ (centered at 495 nm). Sensitized excitation of the phosphorylated 5-FAM-SFAStide-A-Tb³⁺ complex through phosphotyrosine triggers energy transfer to 5-FAM, giving emission from 5-FAM at its characteristic wavelength (˜520 nm), which falls in a relatively “empty” region of the Tb³⁺ emission spectrum (FIG. 1B). Similarly, detection of pSAStide-Cy5-Tb³⁺ complex is achieved based on the overlap of the Cy5 excitation band with the ⁵D₄→⁷F₄ and ⁵D₄→⁷F₃ emission bands of Tb³⁺ centered at 595 nm and 620 nm, giving Cy5 emission at its characteristic wavelength (˜670 nm) which is also free of interference from Tb³⁺ emission (FIG. 1C).

Suitable fluorophores that can be incorporated into the peptides used in the methods of the invention include fluorophores comprising the core structure of coumarin, hydroxyphenylquinazolinone (HPQ), dicyanomethylenedihydrofuran (DCDHF), fluorescein, rhodol, rhodamine, rosamine, boron-dipyrromethene (BODIPY), resorufin, acridinone, or indocarbocyanine, or analogs thereof. Other suitable fluorophores that can be incorporated into the peptides include quantum dots. Additional fluorophores that can be incorporated into the peptides include the fluorophores discussed at Wysocki and Lavis, Current Opinion in Chemical Biology, 15, 752-759 (2011); Resch-Genger et al, Nature Methods, 5, 763-775 (2008); Mashinchian et al, BioImpacts, 4, 149-166 (2014); Chozinski et al, FEBS Letters, 588, 3603-3612 (2014); Umezawa et al, Analytical Sciences, 30, 327-349 (2014); Zheng et al, Chem Soc Rev, 43, 1044-1056 (2014); and Terai and Nagano, Pflugers Arch—Eur J Physiol 465, 347-359 (2013); www.fluorophores.tugraz.at-/substance/ and www.biosyn.com/Images/ArticleImages/Comprehensive-%20fluorophore%20list.pdf.

Other suitable fluorophores include fluorescent proteins that have an excitation wavelength overlap with one of the emission bands of at least one of the lanthanides, such as the fluorescent proteins disclosed at Olenych et al, Current Protocols in Cell Biology, Ch. 21, Unit 21.5, (2007); Enterina, Wu and Campbell, Current Opinion in Chemical Biology, 27, 10-17 (2015); and Shaner et al, J. Cell Science, 120, 4247-4260 (2007).

Specific fluorophores that can be incorporated into the peptides include GFP, EGFR, RFP, ERFP, mPlum, mCherry, 5-FAM, tetramethylrhodamine, Alexafluor-488, Alexafluor-555, Alexafluor-680, DyLight-488, DyLight-550, Cy3, and Cy5. More specific fluorophores suitable for use in the invention, include 5-FAM and Cy5.

In one embodiment, each peptide is linked to a fluorophore through a direct bond or a linking group.

### Linking Group

The structure of the linking group is not critical provided the resulting linked peptide is capable of functioning in the methods of the invention.

In one embodiment the linking group has a molecular weight of from about 20 daltons to about 1,000 daltons.

In one embodiment the linking group has a molecular weight of from about 20 daltons to about 200 daltons.

In another embodiment the linking group has a length of about 5 angstroms to about 60 angstroms.

In another embodiment the linking group separates the chelating group from the remainder of the peptide by about 5 angstroms to about 40 angstroms.

In another embodiment the linking group is a divalent, branched or unbranched, saturated or unsaturated, hydrocarbon chain, having from 2 to 25 carbon atoms, wherein one or more (e.g. 1, 2, 3, or 4) of the carbon atoms is optionally replaced by (—O—) or (—NH—), and wherein the chain is optionally substituted on carbon with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In another embodiment the linking group is a divalent, branched or unbranched, saturated or unsaturated, hydrocarbon chain, having from 2 to 25 carbon atoms, wherein one or more (e.g. 1, 2, 3, or 4) of the carbon atoms is optionally replaced by (—O—), and wherein the chain is optionally substituted on carbon with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In another embodiment the linking group is a divalent, branched or unbranched, saturated or unsaturated, hydrocarbon chain, having from 2 to 10 carbon atoms.

In another embodiment the linking group is a divalent, branched or unbranched, saturated hydrocarbon chain, having from 2 to 10 carbon atoms.

In another embodiment, the linking group comprises a binding pair.

In another embodiment, the “binding pair” refers to two molecules which interact with each other through any of a variety of molecular forces including, for example, ionic, covalent, hydrophobic, van der Waals, and hydrogen bonding, so that the pair have the property of binding specifically to each other. Specific binding means that the binding pair members exhibit binding to each other under conditions where they do not bind to another molecule. Examples of binding pairs are biotin-avidin, hormone-receptor, receptor-ligand, enzyme-substrate, IgG-protein A, antigen-antibody, and the like.

In another embodiment, a first member of the binding pair comprises avidin or streptavidin and a second member of the binding pair comprises biotin.

In another embodiment, one member of a binding pair is covalently linked, either directly or through a linking group, to a peptide that is a substrate for a kinase and the other member of the binding pair is associated (e.g. covalently bonded directly or through a linking group or associated through any of a variety of molecular forces) with a quantum dot.

In another embodiment, one member of a binding pair is covalently linked, either directly or through a linking group, to a peptide that is a substrate for a kinase and the other member of the binding pair is covalently linked, either directly or through a linking group, to a quantum dot.

In another embodiment, each peptide that is a substrate for a kinase is covalently linked, either directly or through a linking group, to a biotin which specifically binds to a streptavidin coated quantum dot.

The invention will now be illustrated by the following non-limiting Examples.

### EXAMPLES

### Example 1

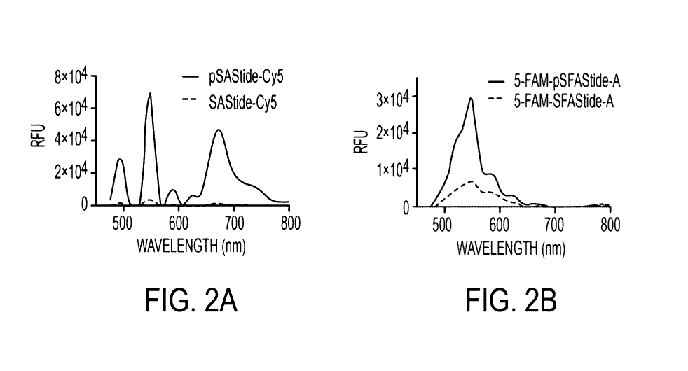

Time-resolved analysis of each peptide biosensor in the presence of Tb³⁺ provided the four characteristic luminescence emission peaks from Tb³⁺ as well as the fluorescence emission peak from the conjugated fluorophore label (FIGS. 2A, 2B). Quantitative comparison of the emission spectra between the phosphorylated and unphosphorylated biosensors showed a 25-fold increase in intensity at the Cy5 emission maximum (λ₆₇₀) for pSAStide-Cy5 (FIG. 2A), and a 3.9-fold increase in intensity at the 5-FAM emission maximum (λ₅₂₀) for 5-FAM-pSFAStide-A (FIG. 2B). Control experiments in the presence and absence of Tb³⁺ showed that excitation of Cy5 was Tb³⁺- and therefore LRET-dependent rather than arising from direct excitation of the fluorophore. 5-FAM showed some low-level background excitation in the absence and presence of Tb³⁺ (FIGS. 7A, 7B and 7C). This did not substantially affect the LRET readout for the 5-FAM-SFAStide-A (since excitation is performed at 266 nm, at which 5-FAM did not show any excitation). These changes in the intensity of the fluorophore signals upon phosphorylation of their respective peptides provide sensor-specific spectral features that can be monitored to determine phosphorylation of the sensors and consequently kinase activity.

### Example 2

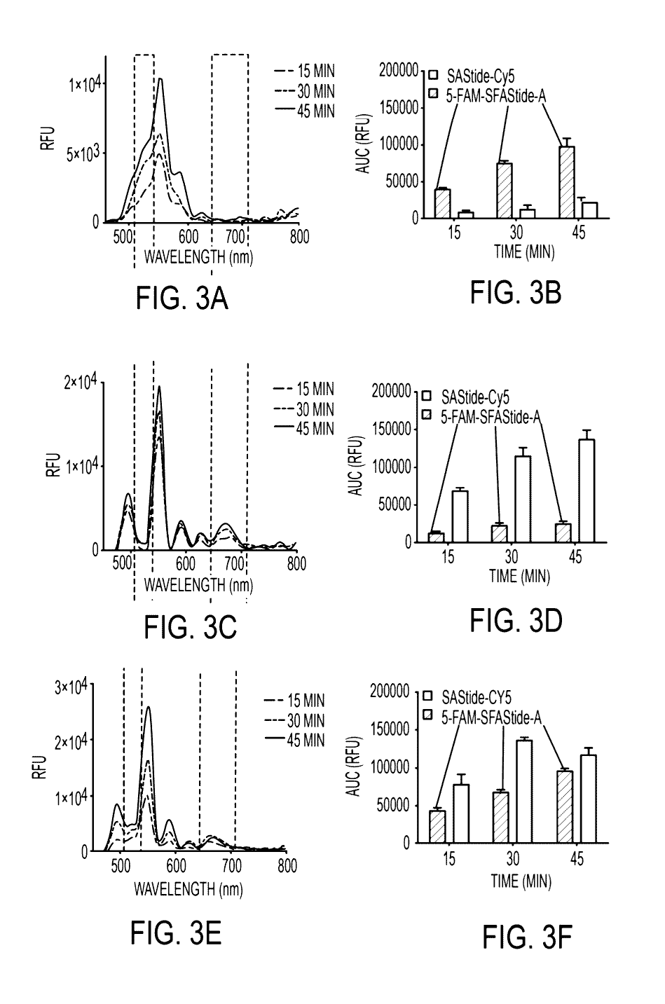

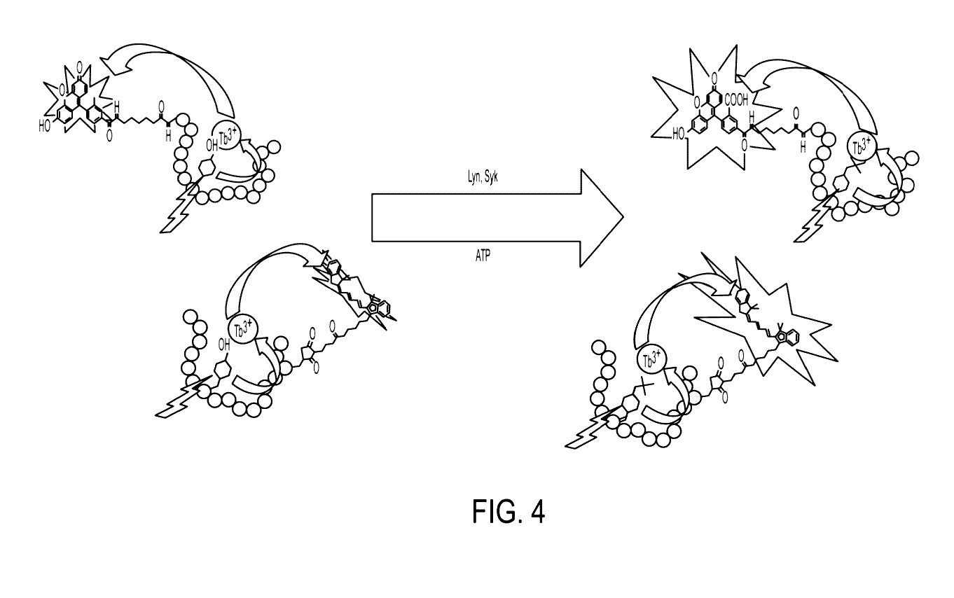

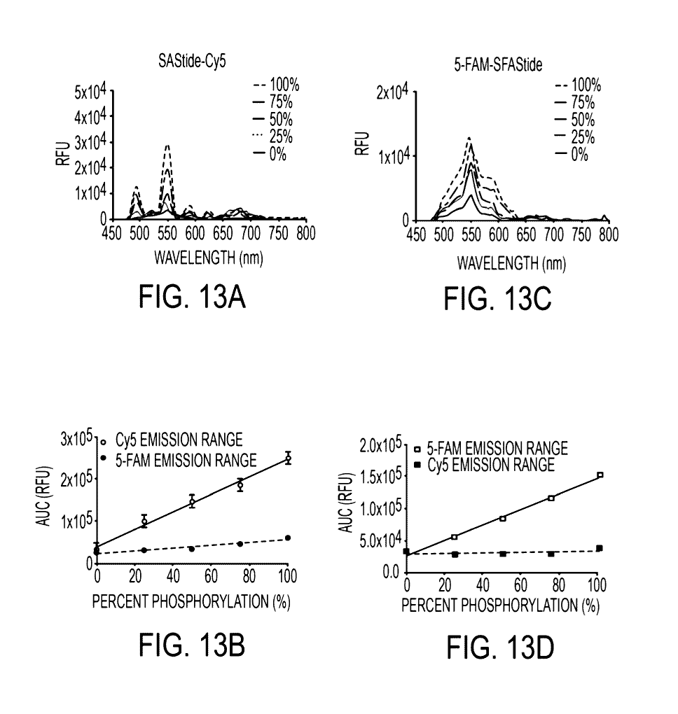

After establishing the relationship between sensor phosphorylation and TR-LRET signal, the two biosensors in a kinase assay were employed. Analysis of Syk and Lyn activities in vitro was accomplished using the purified kinases with the kinase reaction buffer and detection conditions described in the supporting information. Briefly, after pre-incubation of the kinases with the reaction buffer for about minutes, the reaction was initiated by the addition of the biosensor(s). Aliquots were removed from the reaction, quenched with urea, treated with Tb³⁺, and brought to a volume of 100 μL. In the presence of only one or the other of the kinases, TR-LRET emission spectra for each respective biosensor displayed an increase in the conjugated dye's fluorescence signal (with minimal bleed through or background interference from the fluorophore attached to the other biosensor) over the time course of the reaction (FIGS. 3A-D). These results confirmed the relative specificity of each biosensor for its individual kinase, in agreement with previously reported results for SAStide and a separate assay using ELISA-based chemifluorescence detection for SFAStide-A (FIG. 13) (Lipchik, A. M., et al., Biochemistry, 2012, 51, 7515). Finally, to demonstrate multiplex detection, both biosensors were incubated with both kinases in a single reaction. A simultaneous increase in intensity for both fluorophores was seen over the time course, indicating an increase in phosphorylation of both peptides (FIG. 3E).

Dual kinase detection was accomplished using the environmentally sensitive fluorophores oxazine and cascade yellow conjugated to peptide substrates for the Lyn and Abl kinases, respectively (Wang, Q., et al., ACS Chem Biol, 2010, 5, 887). Unfortunately, most environmentally-sensitive fluorophores are limited in their application in more complex or higher throughput systems by small dynamic ranges and problems with background fluorescence.

The invention provides a novel platform of multiplex detection for the simultaneous monitoring of at least two tyrosine kinase activities, such as, for example (Lyn and Syk) using a Src-Family kinase Artificial Substrate peptide (SFAStide) and SAStide (Sky Artificial Substrate peptide) (sequences shown in Table 1) (Lipchik, A. M., Parker, L. L., Anal Chem, 2013, 85, 2582; Lipchik, A. M., et al., J Am Chem Soc, 2015, 137, 2484). Multi-colored detection is achieved through time-resolved luminescence energy transfer (TR-LRET) by employing the kinase specific phosphopeptide-Tb³⁺ complexes as the energy donors and the conjugated fluorophores as the energy acceptors. As a non-limiting example, cyanine 5 (Cy5) and 5-carboxyfluorescein (5-FAM) can serve as the donor and acceptor, respectively.

### Example 3A

### Peptide Synthesis

Peptides SAStide (GGDEEDYEEPDEPGGCGG), pSAStide (GGDEEDYEEPDEPGGCGG), 5-FAM-SFAStide-A (5-FAM-Ahx-GGEEDEDIYEELDEPGGK_{biotin}GG) and 5-FAM-pSFAStide-A (5-FAM-Ahx-GGEEDEDIYEELDEPGGK_{biotin}GG) were synthesized as previously described, by Lipchik, A. M., et al., J Am Chem Soc, 2015, 137, 2484, on a 50 μmol scale using a Protein Technologies Prelude Parallel peptide synthesizer on MBHA-amide resin (Peptides International). Coupling of standard Fmoc (9-fluorenylmethoxy-carbonyl)-protected amino acids (4 equiv)(Peptides International) were achieved with HCTU (3.8 equiv) in the presence of NMM (8 equiv) in DMF for two 10 min couplings. Fmoc deprotection was achieved in 20% piperidine in DMF for two 2.5 min cycles. Side-chain deprotection and peptide cleavage from the resin was performed in 5 ml cocktail of trifluoroacetic acid (TFA):water:ethane dithiol (EDT):triisopropylsilane (TIS) (94:2.5:2.5:1). Peptides were precipitated and washed three times with cold diethyl ether. The peptides were dissolved in acetonitrile:water: TFA (50:50:0.1), flash frozen and lyophilized. The peptides were purified by preparative reverse-phase HPLC (Agilent Technologies 1200 Series) a using C18 reverse-phase column. Peptides were characterized by LCMS and MALDI-TOF analysis.

SAStide was labeled with AlexaFluor-488-maleimide (Invitrogen) or Cy5-maleimide (Lumiprobe) in TCEP and 100 mM phosphate buffer at pH 6.5. Reaction progress was monitored by MALDI-TOF MS and was found to be complete after 2 h. The labeled peptide was purified using a C18 cartridge (50 mg, Waters) and lyophilized. The labeled peptides were then characterized by LC/MS analysis.

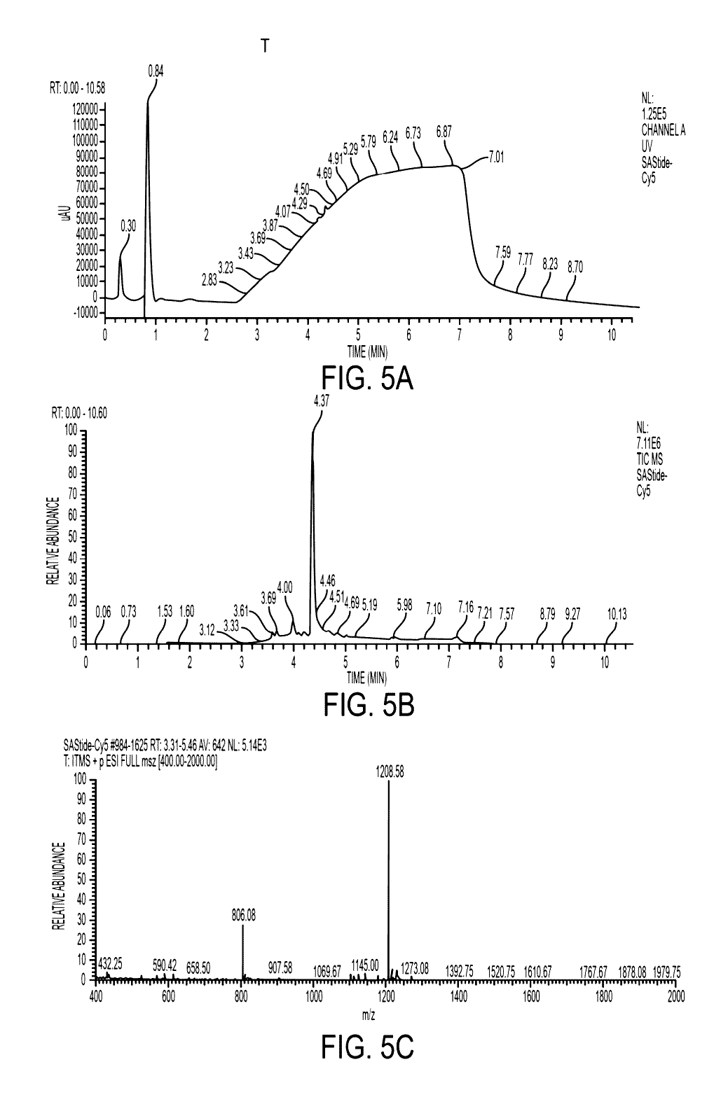

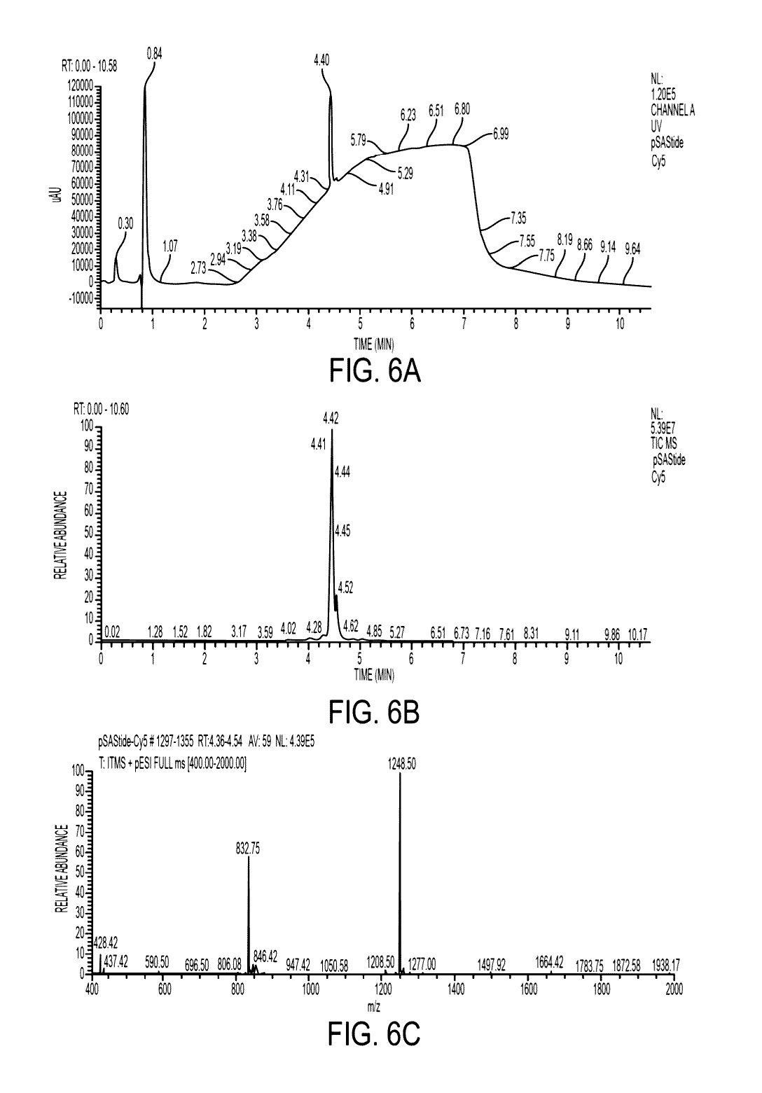

The peptides were characterized using molecular weight analysis, Mass spec, Cy5 absorbance, and UV spectroscopy. FIGS. 5A, 5B, and 5C, are for SAStide-Cy5 (GGDEEDYEEPDEPGGC_{Cy5G}G); FIGS. 6A, 6B, and 6C, are for pSAStide-Cy5 (GGDEEDYEEPDEPGGC_{Cy5}GG); FIGS. 7A, 7B, and 7C, are for 5-FAM-SFAStide-A (5-FAMAhxGGEEDEDIYEELDEPGGK_{biotin}GG); and FIGS. 8A, 8B, and 8C, are for 5-FAM-pSFAStide-A (5-FAMAhxGGEEDEDIYEELDEPGGK_{biotin}GG).

### Example 3B

### Peptide Concentration

Peptides were dissolved in distilled water and diluted using 20 mM Tris buffer, pH 9.0. UV spectroscopy of 5-FAM, AF488, or Cy5 absorbance was determined and the concentration of the peptide solution was calculated according to Beer's Law.

### Example 3C

### Absorbance of SAStide-Cy5, pSAStide-Cy5, 5-FAM-SFAStide-A and 5-FAM-pSFAStide-A Tb³⁺ Complexes

The UV absorbance spectra of SAStide-Cy5 in its phosphorylated and unphosphorylated form each displayed a single absorbance band. 5-FAM-SFAStide-A showed two absorbance maxima, one for the tyrosine and the other presumably related to the 5-FAM fluorophore (since it was present both with and without Tb³⁺).

### Example 4

### Luminescence Emission Measurements

Time-resolved emission spectra were collected on a Biotek Synergy4 plate reader at room temperature in black 384-well plates (Greiner Fluortrac 200). Spectra were collected from 450-800 nm after excitation at 266 nm with a delay time of 50 μsec and a gate time of 1 msec. Sensitivity (an instrument parameter similar to gain) was adjusted as necessary and is reported where relevant.

### Example 5

### In Vitro Kinase Assay

Assays were performed as previously described in Lipchik, A. M., Parker, L. L. Anal Chem. 2013, 85, 2582. His₆-tagged Syk was isolated from HEK293 cells stably expressing Syk-His₆. Cells were lysed using Phosphosafe extraction buffer (Novagen) containing protease inhibitor cocktail (Roche). Syk-His₆ was purified using Ni²⁺ magnetic bead, washed with kinase reaction buffer and eluted with 1 M imidazole. (Promega). The concentration of Syk was determined by BCA protein assay (Pierce). Syk-His₆ and/or Lyn was incubated with the kinase reaction buffer (100 μM ATP, 10 mM MgCl₂, 12.5 μg/μL BSA and HEPES pH 7.5) containing SAStide-Cy5 and 5-FAM-SFAStide-A at 12.5 μM and 2.5 μM respectively at 30° C. Aliquots were taken at designated time points and quenched in 20 μL 6 M Urea. The quenched samples were then used for detection using terbium luminescence in the presence of 10 μL 100 μM Tb³⁺.

### Example 6

### Luminescent Properties of pSAStide-Cy5 and 5-FAM-pSFAStide-A

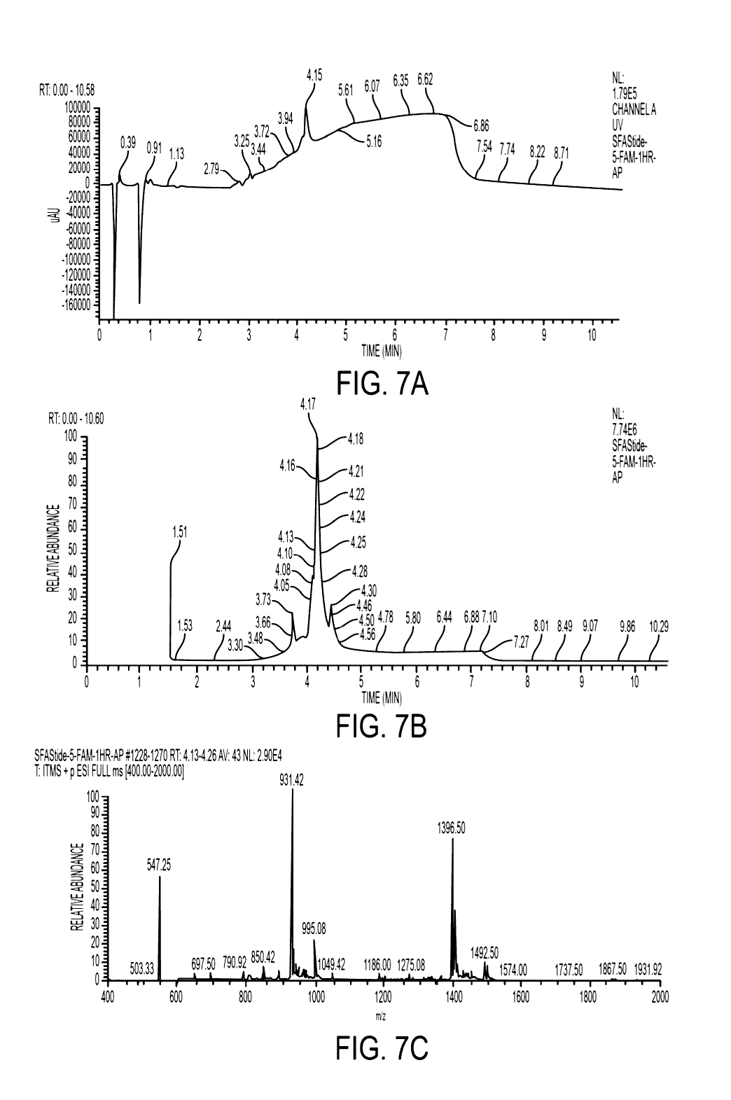

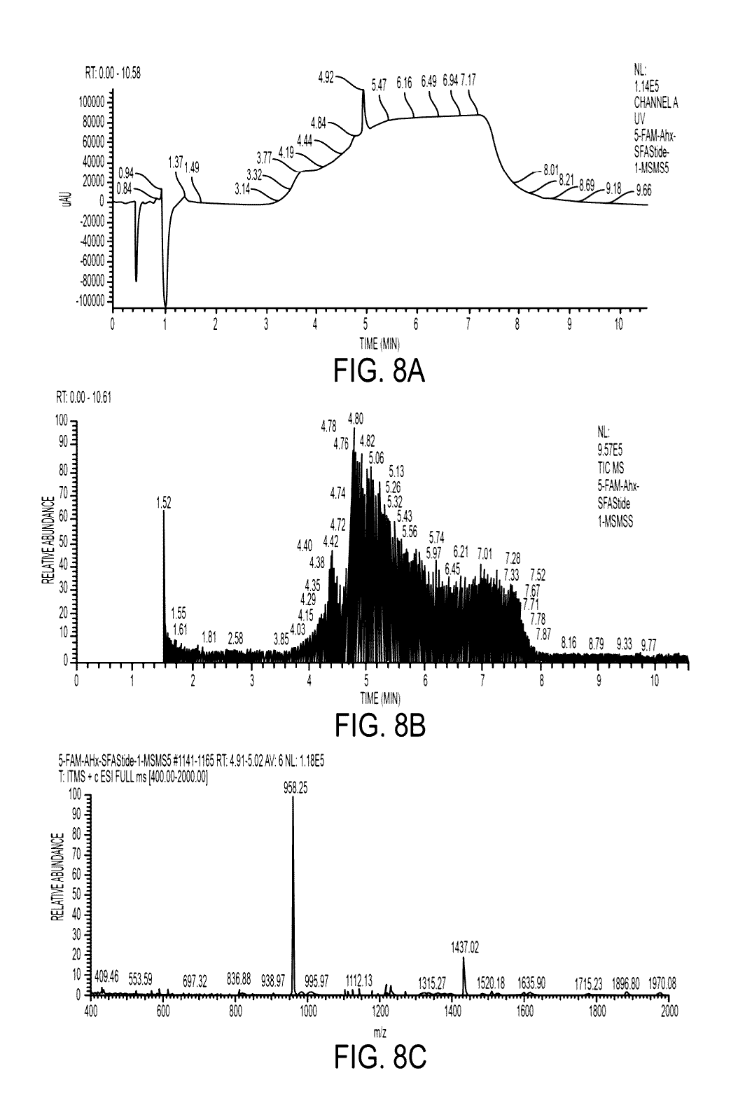

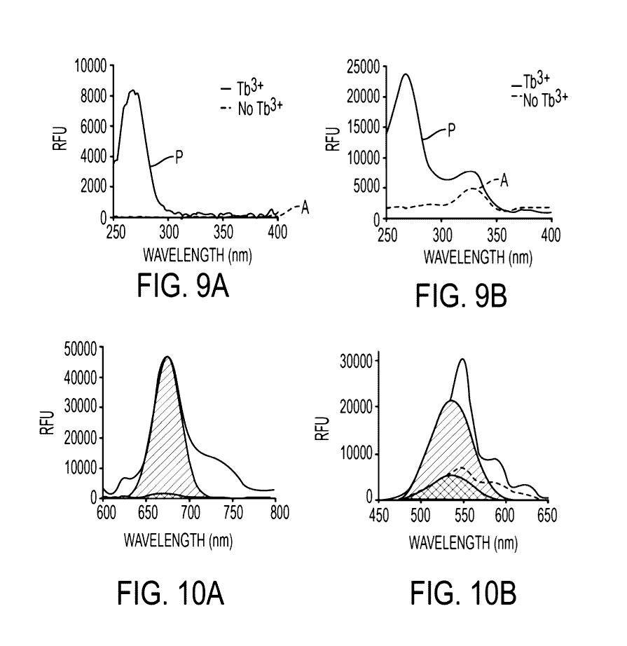

Luminescence excitation spectra for pSAStide-Cy5, illustrated in FIG. 9, (9A) and 5-FAM-pSFAStide-A (9B) were collected in the presence (P) or absence (A) of Tb³⁺. Emission at the respective λₘₐₓ for each organic fluorophore (Y-axis) was measured at the excitation wavelengths across the range for tyrosine absorbance (shown on the X-axis). While Cy5 showed no excitation in the absence of Tb³⁺ (indicating complete Tb³⁺-dependence), 5-FAM showed some background excitation both in the absence and presence of Tb³⁺, however at a higher wavelength than is used in the typical LRET biosensor assay (266 nm). Emission maxima were collected from 15 μM peptide in the presence of 100 μM Tb³⁺ or absence, 10 mM HEPES, 100 mM NaCl with a 50 μs delay and 1 ms collection time. Each spectrum represents the average of three replicates.

### Example 7

### Quantification of Cy5 and 5-FAM Fluorescence Intensity

Quantification of the fluorophore signal was accomplished for SAStide-Cy5 (A) and 5-FAM-SFAStide-A (B) by fitting a Gaussian curve to the individual signals and integrating the curve. Results are illustrated in FIGS. 10A and 10B.

### Example 8

### Validating Lack of Interference in Dualplexed Detection

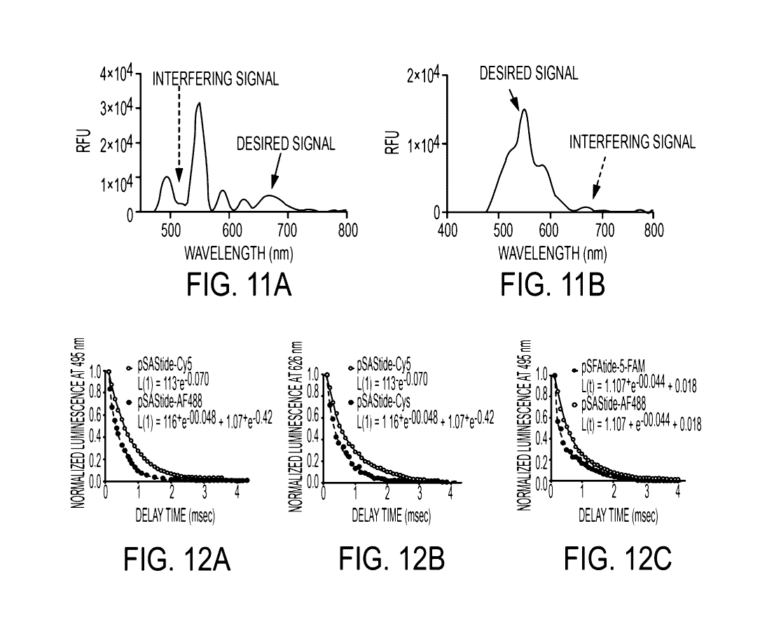

The conditions were initially optimized using phosphorylated SAStide sensor (pSAStide-Cy5) with unphosphorylated SFAStide-A-5-FAM peptide. Adjusting the concentration of SFAStide-A, increasing the delay time, and varying the concentration of the Tb³⁺ successfully mitigated any interference from the 5-FAM signal caused by intermolecular LRET (FIG. 11A) Using the same conditions, the cross-interference from SAStide-Cy5 was examined and minimized in the presence of pSFAStide-A-5-FAM (FIG. 11B).

FIG. 11A, pSAStide-Cy5 cross-interference with SFAStide-A-5-FAM signal and FIG. 11B, pSFAStide-A-5-FAM cross-interference with SAStide-Cy5 signal. Spectra were collected from 0.5 μM SFAStide-A-5-FAM and 2.5 μM SAStide-Cy5 in the presence of 10 μM Tb³⁺ in 10 mM HEPES, 100 mM NaCl, pH 7.5, 1.2 M Urea, 20 μM ATP, 0.2 ng/μL BSA, 2 mM MgCl₂, λₑₓ=266 nm, in 100 μl total volume, 1 ms collection time, 100 μs delay time, and sensitivity 180. Data represent the average of experiments performed in triplicate.

### Example 9

### Determination of LRET Distance

The distance between the Tb³⁺ ion and the fluorophore is a critical parameter for energy transfer, in which the intensity of the acceptor fluorescence signal displayed in the emission spectrum is directly related to the optimal distance. The Tb³⁺ luminescence lifetimes of the biosensors in their fluorophore conjugated and unconjugated forms were used to characterize the energy transfer and LRET parameters for each sensor (FIGS. 12A, 12B and 12C). LRET follows the same principles as FRET and can have the same theory applied to calculate the distance between the fluorophore acceptor and the terbium-peptide complex donor pair. The fundamental concept of Förster theory is resonance energy transfer is proportional to

R=R₀[(1/E)−1]^{1/6} (S1)

where the percentage of energy transfer, E, can be determined from the lifetime measurements of the donor in the absence of the acceptor (peptide-terbium complex (donor) without the conjugated fluorophore (acceptor)) and the donor in the presence of the acceptor.

$$E = \frac{1 - {τ}_{DA}}{{τ}_{D}} \qquad ( S2 )$$

R₀ the Förster distance is determined for each acceptor/donor pair and d

R₀=0.211(κ²η⁻⁴Q_{D}J) (S3)

Where k2 is the J is determined by the following equation

$$J = \frac{\sum [ {F}_{D} ( λ ) ɛ ( λ ) {λ}^{4} Δ λ ]}{\sum [ {F}_{D} ( λ ) Δ λ ]} \qquad ( S4 )$$

The luminescence decay rates peptide biosensor-Tb³⁺ complexes with and without fluorophore conjugation are illustrated in FIG. 12A pSAStide-AF488:Tb³⁺, FIG. 12B pSAStide-Cy5 and FIG. 12C 5-FAM-pSFAStide-A. Data represent the average±SEM of three individual replicates.

TR-LRET measurements showed that energy transfer from Tb³⁺ to the various fluorophores was very efficient (in the range of 89-93%). The radius representing the estimated distance between Tb³⁺ and the fluorophore on the peptide, R, and the Förster radius, R₀, ranged from 50-55 Å and 35-40 Å, respectively, which, as indicated by the efficient energy transfer, are within the optimal range for TR-LRET measurements. See, Vogel, K. W.; Vedvik, K. L., J Biomol Screen 2006, 11, 439. SAStide was also conjugated with AlexaFluor 488 (AF488) as an additional control for the measurements to demonstrate the agreement in LRET parameters when using different fluorophores.

TABLE 2
LRET Data for Calculating Distance

| | | | Quan- | | | | |
|---|---|---|---|---|---|---|---|
| | τ_{D} | τ_{DA} | tum | Rₒ | J (M⁻¹ cm⁻¹ | Energy | R |
| Biosensor | (ms) | (ms) | Yield | (Å) | nm⁴) | Transfer | (Å) |
| SAStide- | 0.70 | 0.048 | 0.34 | 58.9 | 8.04 × 10¹⁴ | 0.93 | 38.2 |
| AF488 | | | | | | | |
| SAStide- | 0.72 | 0.088 | 0.34 | 55.1 | 5.4 × 10¹³ | 0.88 | 39.5 |
| Cy5 | | | | | | | |
| 5-FAM- | 0.64 | 0.071 | 0.21 | 56.0 | 9.62 × 10¹⁴ | 0.89 | 39.5 |
| SFAStide- | | | | | | | |
| A | | | | | | | |

### Example 10

### Calibration Curve for Increasing Amount of Phosphorylation

Time-resolved analysis of each peptide biosensor in the presence of Tb³⁺ gave the four characteristic luminescence emission peaks from Tb³⁺ as well as the fluorescence emission peak from the conjugated fluorophore label (FIGS. 2A, 2B). Quantitative comparison of the emission spectra between the phosphorylated and unphosphorylated biosensors showed a 25-fold increase in intensity at the Cy5 emission maximum (λ₆₇₀) for pSAStide-Cy5 (FIG. 2A), and a 3.9-fold increase in intensity at the 5-FAM emission maximum (λ₅₂₀) for 5-FAM-pSFAStide-A (FIG. 2B). Control experiments in the presence and absence of Tb³⁺ showed that excitation of Cy5 was Tb³⁺- and therefore LRET-dependent rather than arising from direct excitation of the fluorophore. 5-FAM showed some low-level background excitation in the absence and presence of Tb³⁺ (FIGS. 7A, 7B, and 7C), but this did not substantially affect the LRET readout for the 5-FAM-SFAStide-A (since excitation is performed at 266 nm, at which 5-FAM did not show any excitation). These changes in the intensity of the fluorophore signals upon phosphorylation of their respective peptides provide sensor-specific spectral features that can be monitored to determine phosphorylation of the sensors and consequently kinase activity.

In order to achieve multiplex detection in the same sample, the reaction and detection conditions needed to be optimized to have limited cross-interference between sensors. Cross-interference was evaluated by analyzing the fluorophore signal from an unphosphorylated sensor in the presence of the other phosphorylated biosensor. To accomplish this, the concentrations of the biosensors and Tb³⁺, as well as the delay time, were varied and TR-LRET spectra collected. Quantification was accomplished by Gaussian fitting of the fluorophore emission peaks and integrating the resulting curves for each peak (see FIG. 8). Under the optimized conditions, the TR-LRET spectra for each phosphorylated biosensor displayed minimal signal from cross-interfering fluorophore, while giving significantly stronger signal for the desired fluorophore (absorbance FIGS. 9A, 9B; quantification FIGS. 10A, 10B). TR-LRET distance parameters were also characterized (below and Table 1).

Next, a calibration curve was plotted to show the quantitative relationship between sensor phosphorylation and its corresponding TR-LRET signal for each sensor (FIGS. 12A, 12B and 12C). Experiments were performed in the presence of the unphosphorylated form of the other biosensor and the kinase reaction buffer (to best mimic the conditions of a multiplexed kinase reaction). Proportion of phosphorylated peptide was quantitatively determined by integrating the signal centered at 520 nm for 5-FAM and 670 nm for Cy5. The high signal to noise ratio observed in the initial control experiments was maintained in the presence of the reaction buffer with 7.6:1 for SAStide-Cy5 and 5.8:1 for 5-FAM-SFAStide-A. Z′-factor and signal window (SW) values were calculated and shown to be appropriate for HTS with Z′-factor values of 0.72 and 0.78, and SW of 13.27 and 12.65, for SAStide-Cy5 and 5-FAM-SFAStide-A, respectively. Details of these calculations are provided herein.

TR-LRET quantitative detection of biosensor phosphorylation. (FIG. 13A) pSAStide-Cy5-Tb³⁺ emission spectra with increasing proportions of phosphorylated biosensor compared to unphosphorylated in the presence of unphosphorylated 5-FAM-SFAStide-A. (FIG. 13B) Cy5 emission spectral area calibration curve based on spectra from (FIG. 13A) and the integrated area of the Cy5 emission peak. (FIG. 13C) 5-FAM-pSFAStide-A-Tb³⁺ emission spectra at increasing proportions of phosphorylated biosensor compared to unphosphorylated in the presence of unphosphorylated SAStide-Cy5. (FIG. 13D) 5-FAM emission spectral area calibration curve based on (FIG. 13C). Spectra were collected from 0.5 μM SFAStide-A-5-FAM and 2.5 μM SAStide-Cy5 in the presence of 10 μM Tb³⁺ in 10 mM HEPES, 100 mM NaCl, pH 7.5, 6 M Urea, 100 μM ATP, 12.5 μg/μL BSA, 10 mM MgCl₂, λₑₓ=266 nm, in 100 μL total volume, 1 ms collection time, 100 μs delay time, and sensitivity 180. Data represent the average of experiments performed in triplicate, error bars in the AUC plots represent SEM.

### Example 11

### Determination of HTS Screening Parameters

The limit of detection (LOD) and the limit of quantification (LOQ) were determined:

LOD=3*σ_{neg}+μ_{neg}

LOQ=10*σ_{neg}+μ_{neg}

where σ_{neg} is the standard deviation of the negative control sample and μ_{neg} is the mean value of the negative control sample.

High-throughput screening parameters were evaluated using the following equation for Z′-factor (from Iverson et al., Eds.; Eli Lilly & Company and the National Center for Advancing Translational Sciences):

$${Z}^{′} = \frac{( {μ}_{pos} - \frac{3 {σ}_{pos}}{\sqrt{n}} ) - ( {μ}_{neg} + \frac{3 {σ}_{neg}}{\sqrt{n}} )}{( {μ}_{pos} - {μ}_{neg} )}$$

and the signal window was calculated by the following equation:

$$SW = \frac{( {μ}_{pos} - \frac{3 {σ}_{pos}}{\sqrt{n}} ) - ( {μ}_{neg} + \frac{3 {σ}_{neg}}{\sqrt{n}} )}{\frac{{σ}_{pos}}{\sqrt{n}}}$$

TABLE 3
HTS assay parameters for SAStide-Cy5 controls

| Percent | CV | Average (Area | Standard | Z | Signal |
|---|---|---|---|---|---|
| Phosphorylation | (%) | 10⁵) | Deviation | factor | Window |
| 0% | 24.29 | 32900 | 13847 | N/A | N/A |
| 25% | 14.33 | 100443 | 24936 | 0.005 | 0.025 |
| 50% | 8.39 | 146455 | 21294 | 0.46 | 4.29 |
| 75% | 6.25 | 186279 | 20175 | 0.62 | 8.11 |
| 100% | 4.74 | 249915 | 20540 | 0.73 | 13.28 |

TABLE 4
HTS assay parameters for 5-FAM-SFAStide-A controls

| Percent | CV | Average (Area | Standard | Z | Signal |
|---|---|---|---|---|---|
| Phosphorylation | (%) | 10⁵) | Deviation | factor | Window |
| 0% | 5.29 | 75198 | 6892 | N/A | N/A |
| 25% | 4.44 | 146636 | 11280 | 0.56 | 6.14 |
| 50% | 14.22 | 235620 | 58039 | 0.30 | 1.43 |
| 75% | 4.28 | 300040 | 22218 | 0.78 | 13.60 |
| 100% | 4.99 | 398544 | 34459 | 0.78 | 12.65 |

### Example 12

### Validation of SAStide and SFAStide-A Phosphorylation and Specificity In Vitro

Phosphorylation of SAStide and SFAStide-A were detected using a chemifluorescent ELISA-based assay (Lipchik et al. J Am Chem. Soc 2015, 137, 2484) in which the reaction mixture was quenched using EDTA and incubated in a 96-well neutravidin coated-plate to allow for affinity capture of the biotinylated substrates individually. The total amount of peptide in the quenched reaction mixture applied to each well was 37.5 pmol, which ensured that each well was saturated with peptide (15 pmol binding capacity) for analysis. The captured peptide was then incubated an anti-phosphotyrosine primary antibody (4G10) followed by a horseradish peroxidase-conjugated secondary antibody. Chemifluorescent detection was accomplished by incubating each well with Amplex Red reagent and hydrogen peroxide in phosphate buffer, which gave a fluorescent signal proportional to the amount of horseradish peroxidase-conjugated antibody in each well, and thus reports the degree of phosphotyrosine present. As seen in the with the Tb³⁺ based detection, the ELISA-based assay displayed increasing fluorescent signal over time for the appropriately match substrates, demonstrating that SAStide-Cy5 was phosphorylated by Syk and 5-FAM-SFAStide-A was phosphorylated by Lyn in vitro.

### Example 13

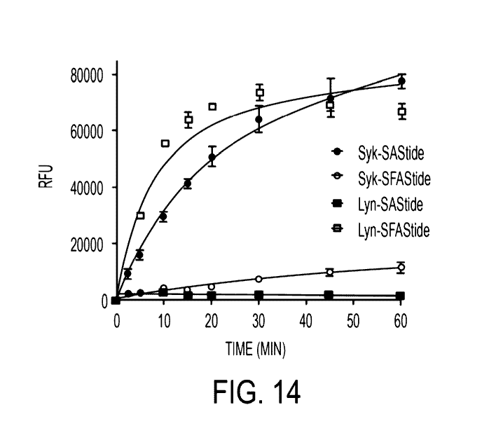

The Validation of in vitro specificity of SAStide-Cy5 and 5-FAM-SFAStide-A using ELISA-based chemifluorescence is illustrated in FIG. 14. The SAStide biosensor was incubated with Syk-EGFP and the 5-FAM-SFAStide-A biosensor with Lyn in an in vitro kinase assay as described in the main text. Aliquots were removed at designated time points, quenched with EDTA and alongside the TR-LRET detection as described in FIGS. 3A-F in the main text, the amount of phosphorylated substrate was also measured using ELISA-based detection.

### Example 14

### Kinase Assay Using Quantum Dot-Peptide Conjugates

### A. Preparation of Quantum Dot-Peptide Conjugates

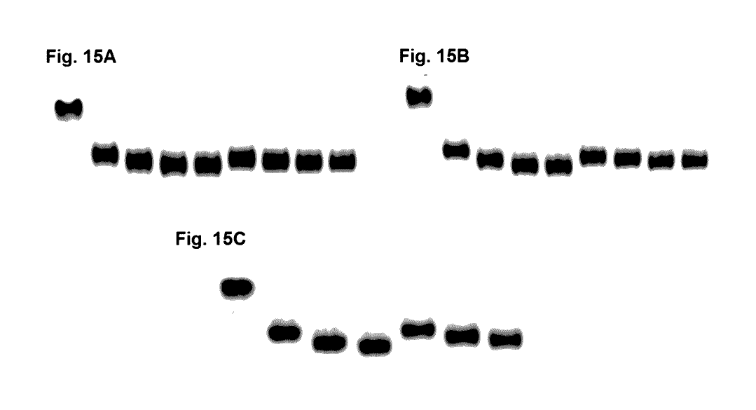

Quantum dot (QD)-peptide conjugates could be readily prepared via the interaction between tetravalent streptavidin and biotin. Streptavidin-coated (Wu, Y. et al. Anal. Biochem. 2007, 364, 193-203) QD605 or QD655 were incubated with SAStide (GGDEEDYEEPDEPGGK_{biotin}GG, a Syk biosensor) (Lipchik, A. M. et al. J Am. Chem. Soc. 2015, 137, 2484-2494) or SFAStide-A (GGEEDEDIYEELDEPGGK_{biotin}GG, a Src family kinase biosensor) (Lipchik, A. M. et al. J. Am. Chem. Soc. 2015, 137, 2484-2494) at room temperature in HEPES buffer (pH=7.5) for 1 hr. To confirm the formation of the conjugates, agarose gel electrophoresis (Ghadiali, J. E. et al. ACS Nano 2010, 4, 4915-4919) (see below) was performed (FIGS. 15A, 15B). QD-biosensor conjugates exhibited increased electrophoretic mobility relative to unlabeled QDs. QDs were saturated (exhibiting no further electrophoretic mobility changes) when biosensor/QD ratio reached about 200:1. Conjugate formation was resistant to high concentration of urea (FIG. 15C), whether the urea addition was prior to or after the incubation process. The QD-biosensor conjugates were also subject to ligand exchange tests, in which the pre-made conjugates showed no signal loss caused by ligand exchange with another labeled QD or excess amount of free biosensors in buffer during a 4 hr incubation, indicating their high stability (deriving from the high affinity of the streptavidin-biotin interaction).

Gel Electrophoresis:

Streptavidin-coated QD605ITK or QD655ITK (2 μM stock solution, Thermo Fisher, USA) and peptides were diluted into 10 mM 2-[4-(2-hydroxyethyl)-piperazin-1-yl]ethanesulfonic acid (HEPES, Calbiochem, USA) buffer (pH=7.5). The final solutions had 10 nM QDs, various amount of peptides, and 2.4 M urea when indicated. After incubation for 1 hr at room temperature, glycerol (100%, Macron Fine Chemicals, USA) was added to each sample with a final concentration of 5% (v/v). The QD-biosensor conjugates were then loaded to a 10 cm long 1% (w/v) agarose (Invitrogen, USA) gels (4 μL sample per well) in 1×TAE buffer (Thermo Fisher Scientific, USA), and run for 60 min at 100 V using BioRad PowerPac Basic power supply. Gels were then imaged using a Gel Logic 112 imaging system (Carestream, USA).

B. Luminescence Emission from QD-Peptide Conjugates

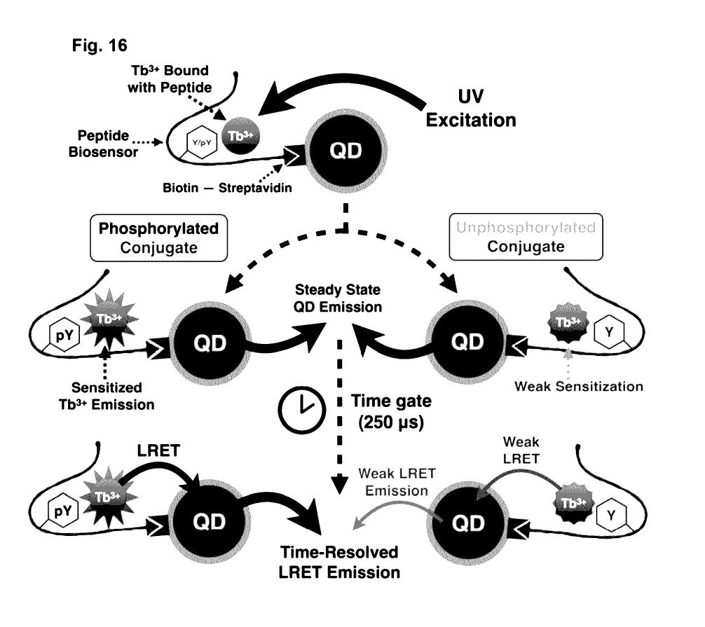

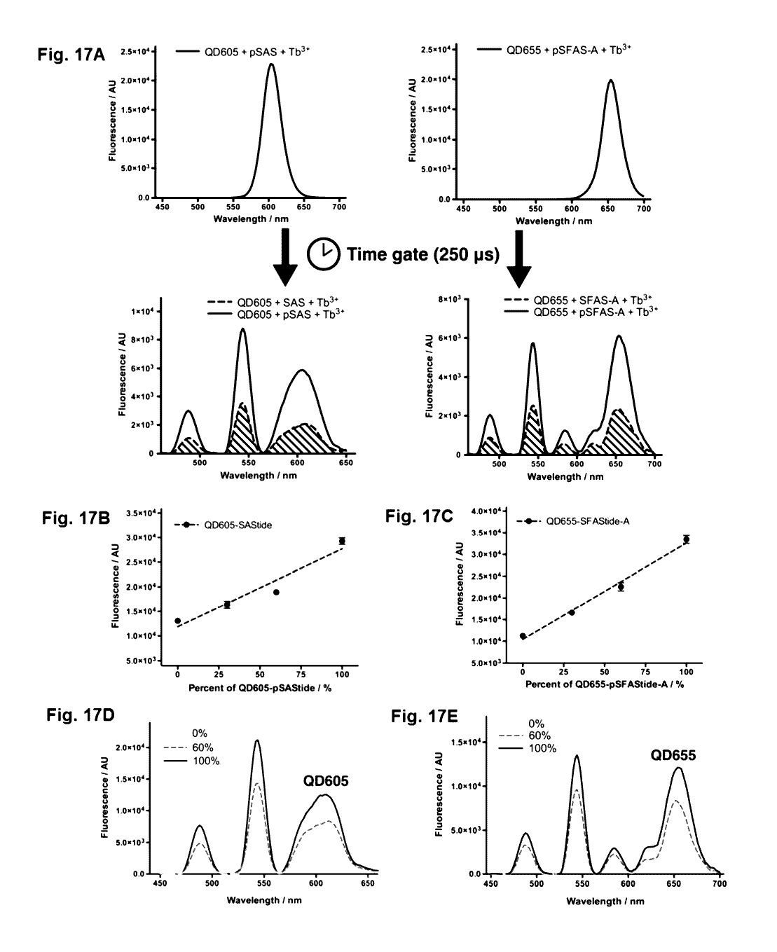

Once the conjugates were prepared, luminescence emission was generated upon UV excitation, from both the QD (short-lived fluorescence) and the peptide-chelated Tb³⁺ (long-lived luminescence) (FIG. 16). Indeed, steady state emission of the conjugates was dominated by inherent QD fluorescence regardless of peptide phosphorylation state as expected (FIG. 17A), given the much higher extinction coefficients of QDs (Wu, Y. et al. Anal. Biochem. 2007, 364, 193-203) (4.4×10⁶ cm⁻¹ M⁻¹ for QD605 at 350 nm and even higher for QD655) than that of Tb³⁺ chelates (Li, M. et al. J. Am. Chem. Soc. 1985, 117, 8132-8138) (˜1.2×10⁴ cm⁻¹ M⁻¹ at 327 nm). However, since QD fluorescence lifetimes are typically less than a hundred nanoseconds, QD inherent emission was greatly reduced by applying a sufficient time gate between excitation and detection (50 μs or longer), while the luminescence emission from Tb³⁺ remained strong. Since Tb³⁺ bound to phosphorylated biosensors had higher luminescence intensity and longer lifetime (Lipchik, A. M. et al. Anal. Chem. 2013, 85, 2582-2588) than the unphosphorylated conjugate, phosphorylation of QD-biosensor conjugates resulted in stronger LRET emission from the QDs (FIG. 17A). Background QD emission in unphosphorylated conjugate samples was low, and was a combined result of residual QD steady-state fluorescence (Morgner, F. et al. Angew. Chem. Int. Ed. Engl. 2010, 49, 7570-7574), free Tb³⁺ binding by buffer components, and unphosphorylated biosensor, which had far lower emission than for the phosphorylated biosensor. Such background emission could be alleviated by optimizing the reaction buffer components/concentrations and instrumental parameters for sufficient delay times. At 10 nM concentration in the reaction buffer, the signal/background for LRET emission from phosphorylated vs. unphosphorylated conjugates was 3:1 when delay time was 250 μs, and could be even higher with longer delay time or higher concentration. Time-resolved emission of both QD605-SAStide and QD655-SFAStide-A conjugates (prepared at biosensor:QD ratio of 100:1 to ensure saturation for the QD surface) showed linear increase with increasing proportion of phosphorylated conjugates, demonstrating that kinase assays using QD-biosensor conjugates can be appropriately quantified according to particular assay conditions and components (FIGS. 17B, 17C, 17D, 17E). The proportion of phosphorylated conjugate was quantified by either the area under curve (AUC) of QD emission peak spectra or by using readings from designated emission filters, and calibration curves were constructed for quantitative detection of the amount of phosphorylation (FIGS. 17B, 17C). Even for proportions of phosphorylated product of as low as 10% (considered the appropriate regime for kinetics experiments), the Z′ factors calculated for both conjugate were >0.5, above the threshold that is generally considered the compatibility requirement for high-throughput screening.

Fluorescence/Luminescence Measurements:

Fluorescence/luminescence emission spectra were measured on a Synergy4 plate reader (Biotek, USA) at room temperature in 384-well black plates (Fluortrac 200, Greiner bio-one, Germany). The emission spectra were collected between 450 and 650 nm with 2 nm increments using the built-in monochromator. Filter-based detection using 550/10, 605/10, 655/10 emission filters (Omega Optical, USA) was also applied when necessary. The excitation wavelength was set to 266 nm by built-in monochromator, or a 265/10 excitation filter (Omega Optical, USA) with 250 μs delay time unless otherwise indicated. Details are described in the Supporting Information.

C. Conjugate Formation Prior to or after the Kinase Reaction

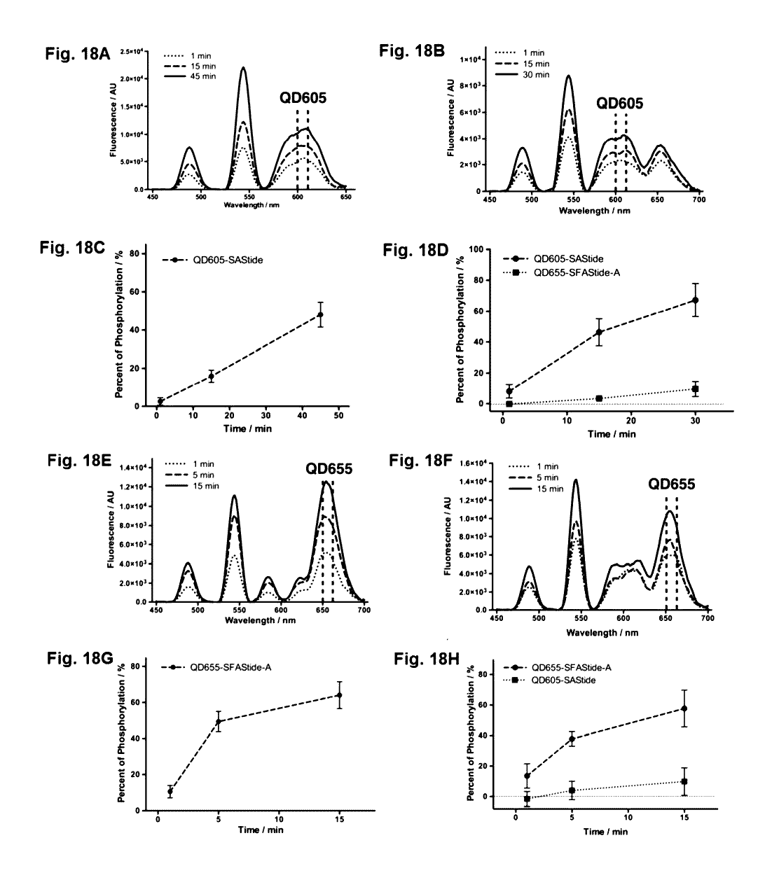

An advantage of the QD-biosensor conjugates shown here is that the TR-LRET kinase assay could be set up with flexible protocols in which the conjugates were prepared either prior to or after the kinase reaction. Briefly, QDs were incubated with the biosensors in HEPES buffer for 1 hr to form the conjugates. Alternatively, QDs could also be added to the quenched samples after the kinase assay. Peptide substrate, as either pre-made QD-biosensor conjugates or free biosensor peptide, was incubated with the reaction buffer (e.g. 15 nM Src, 5 μM peptides, 100 μM adenosine triphosphate (ATP), 10 mM MgCl₂, 0.2 μg/μL BSA, 25 mM HEPES, pH 7.5) for 5 min, and the reaction was started by addition of kinase(s). At selected time points, 40 μL aliquots of the reaction mixture were taken out and quenched in Tb³⁺-containing detection buffer (40 μL 6M urea, 10 μL 1 mM TbCl₃ and 10 μL 1M NaCl) to a final volume of 100 μL. Time-resolved, quantitative readout was done by either monochromator-based spectral scan or filter-based detection using a Biotek Synergy4 plate reader. In the single conjugate kinase assay, TR-LRET analysis of each conjugate displayed a corresponding increase in QD emission signal as a result of biosensor phosphorylation (FIGS. 18A, 18C, 18E, 18G). Kinase activity was readily reported by both the conjugate formed prior to the reaction (FIGS. 18E, 18G) and after the reaction (when the quenched aliquot was incubated with QD to capture biotinylated peptide) (FIGS. 18A, 18C), showing that a QD conjugate strategy could be easily adapted to rapidly changing assay needs without prior chemical preparations such as covalent fluorophore labeling.

### D. Multiplexed Detection of Kinase Activity

For multiplexed detection, QD605-SAStide and QD655-SFAStide-A conjugates were both prepared prior to the assay and then combined in reaction buffer before kinases were added (FIGS. 18B, 18D, 18F, 18H). TR-LRET spectra for the conjugate mixture were collected by the same method above. To minimize the cross interference caused by the overlap of QD605 background emission with QD655 absorption, delay time for QD605-SAStide detection was increased to 400 μs. In the presence of either Src or Syk, both conjugates showed increasing LRET emission corresponding to the specific respective kinase (FIGS. 18B, 18D, 18F, 18H) used over the course of the reactions. Selective phosphorylation of QD605-SAStide by Syk (FIGS. 18B, 18D) and selective phosphorylation of QD655-SFAStide-A by Src (FIGS. 18F, 18H) were demonstrated in the mixture of both conjugates, and the result was in agreement with our previously published work on these two substrates (Lipchik, A. M. et al. Anal. Chem. 2013, 85, 2582-2588 and Lipchik, A. M. et al. J. Am. Chem. Soc. 2015, 137, 2484-2494). TR-LRET emission can be quantified in percent of phosphorylation for either single conjugate assay (FIGS. 18C, 18G) or multiplexed assay (FIGS. 18D, 18H) using corresponding calibration curves (FIGS. 17B, 17C). These results demonstrated the feasibility of using QD-biosensor conjugates in multiplexed TR-LRET kinase assays with a robust readout and flexible procedures.

### E. Advantages of QD-Peptide Conjugate Assay

This kinase assay is inexpensive to produce and does not require any labeling other than standard incorporation of biotinyl-Lysine during their synthesis, the strategy reported here enables flexible combinations of QD/biosensor LRET pairs for assaying a broad, expandable list of kinases (Lipchik, A. M. et al. J. Am. Chem. Soc. 2015, 137, 2484-2494) in a cost efficient manner. Additionally, this approach is more modular, since the choice of LRET donor and acceptor can be decided immediately before the assay without any special chemical labeling planned ahead. The simple nature of this antibody-free assay also renders it more adaptable to the rapidly changing needs of new assay targets, and thus it could be a valuable tool for drug discovery efforts.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A method for detecting the activities of two or more kinases comprising:
a) contacting a first kinase and a second kinase with a first peptide and a second peptide, wherein:
i) the first peptide is a substrate for the first kinase;
ii) the second peptide is a substrate for the second kinase;
iii) each peptide is associated with a lanthanide;
iv) each peptide comprises a group capable of sensitizing the lanthanide that is associated with that peptide; and
v) each peptide is linked to a fluorophore
under conditions such that a first signal associated with the activity of the first kinase and a second signal that is associated with the activity of the second kinase are generated; and
b) detecting the first signal and the second signal.

2. The method of claim 1 wherein each kinase is selected from the group consisting of tyrosine kinases, serine kinases and threonine kinases.

3. The method of claim 1 wherein each kinase is selected from the group consisting of Src-family kinases, Abl-family kinases, and Syk-family kinases.

4. The method of claim 1 wherein each kinase is selected from the group consisting of, Lyn, Syk, and Btk.

5. The method of claim 1 wherein at least one of the peptides is associated with a lanthanide through hydrostatic interactions.

6. The method of claim 1 wherein at least one of the peptides is associated with a lanthanide through a chelating group that is bonded or linked to the peptide.

7. The method of claim 1 wherein each lanthanide is independently selected from the group consisting of La, Ce, Pr, Nd, Pm, Sm, Eu, Gd, Tb, Dy, Ho, Er, Tm, Yb, and Lu.

8. The method of claim 7 wherein each lanthanide is independently selected from the group consisting of Tb, Eu, Sm, Dy, and Yb.

9. The method of claim 8 wherein at least one lanthanide is Tb.

10. The method of claim 1 wherein each group capable of sensitizing the lanthanide comprises an aryl ring or a heteroaryl ring.

11. The method of claim 1 wherein each group capable of sensitizing the lanthanide comprises a phenyl ring.

12. The method of claim 1 wherein each peptide comprises the amino acid tyrosine or tryptophan.

13. The method of claim 1 wherein each peptide comprises the amino acid tyrosine.

14. The method of claim 1 wherein each fluorophore is selected from the group consisting of fluorophores comprising the core structure of coumarin, hydroxyphenylquinazolinone (HPQ), dicyanomethylenedihydrofuran (DCDHF), fluorescein, rhodol, rhodamine, rosamine, boron-dipyrromethene (BODIPY), resorufin, acridinone, or indocarbocyanine, or an analog thereof.

15. The method of claim 1 wherein each fluorophore is selected from the group consisting of GFP, EGFR, RFP, ERFP, mPlum, mCherry, 5-FAM, tetramethylrhodamine, Alexafluor-488, Alexafluor-555, Alexafluor-680, DyLight-488, DyLight-550, Cy3, and Cy5.

16. The method of claim 1 wherein the fluorophore is a quantum dot.

17. The method of claim 1 wherein each peptide is linked covalently either directly or through a linking group to the fluorophore that can be sensitized by the lanthanide metal.

18. The method of claim 17 wherein the linking group is a divalent, branched or unbranched, saturated or unsaturated, hydrocarbon chain, having from 2 to 25 carbon atoms, wherein one or more (e.g. 1, 2, 3, or 4) of the carbon atoms is optionally replaced by (—O—) or (—NH—), and wherein the chain is optionally substituted on carbon with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

19. The method of claim 17 wherein the linking group comprises a binding pair.

20. The method of claim 19 wherein the binding pair is selected from the group consisting of biotin-avidin, hormone-receptor, receptor-ligand, enzyme-substrate, IgG-protein A, antigen-antibody.

21. The method of claim 19 wherein one member of the binding pair is covalently linked, either directly or through a linking group, to each peptide and the other member of the binding pair is associated (e.g. covalently bonded directly or through a linking group or associated through any of a variety of molecular forces) with a quantum dot.

22. The method of claim 19 wherein one member of the binding pair is covalently linked, either directly or through a linking group, to each peptide and the other member of the binding pair is covalently linked, either directly or through a linking group, to a quantum dot.

23. The method of claim 1 wherein each peptide is covalently linked, either directly or through a linking group, to a biotin which specifically binds to a streptavidin coated quantum dot.

24. The method of claim 1 wherein the first signal and the second signal are detected by fluorescence or luminescence spectroscopy.

25. The method of claim 1 wherein the first signal and the second signal are detected by time-resolved fluorescence or time-resolved luminescence spectroscopy.


SEQUENCE LISTING (5 sequences)

SEQ ID NO: 1 - protein, 18 aa, Artificial Sequence
  Description of Artificial Sequence Synthetic peptide
GGDEEDYEEPDEPGGCGG

SEQ ID NO: 2 - protein, 21 aa, Artificial Sequence
  Description of Artificial Sequence Synthetic peptide
XGGEEDEDIYEELDEPGGKGG

SEQ ID NO: 3 - protein, 18 aa, Artificial Sequence
  Description of Artificial Sequence Synthetic peptide
GGDEEDYEEPDEPGGCGG

SEQ ID NO: 4 - protein, 6 aa, Artificial Sequence
  Description of Artificial Sequence Synthetic 6xHis tag
HHHHHH

SEQ ID NO: 5 - protein, 17 aa, Artificial Sequence
  Description of Artificial Sequence Synthetic peptide
GGDEEDYEEDEPGGCGG